(19) United States
(12) Patent Application Publication
(10) Pub. No.: US 2025/0017824 A1
(43) Pub. Date: Jan. 16, 2025

(54) PHAGE DISPENSING SYSTEM

(71) Applicant: Adaptive Phage Therapeutics, LLC, Gaithersburg, MD (US)

(72) Inventors: Rob COHEN, Kensington, MD (US); Greg MERRIL, Bethesda, MD (US); Miguel BARRETO-SANZ, Lausanne (CH)

(21) Appl. No.: 18/596,934

(22) Filed: Mar. 6, 2024

Related U.S. Application Data
(63) Continuation of application No. PCT/US2022/076284, filed on Sep. 12, 2022.
(63) Continuation-in-part of application No. 18/535,422, filed on Dec. 11, 2023.
Application No. 18/535,422 is a continuation of application No. 16/896,718, filed on Jun. 9, 2020, now Pat. No. 11,911,345.
(60) Provisional application No. 63/243,513, filed on Sep. 13, 2021.
(60) Provisional application No. 62/597,008, filed on Dec. 11, 2017.

(51) Int. Cl. A61J 7/00; A61J 1/06; A61K 35/76; B65G 1/137; B65G 61/00; G16H 10/40; G16H 20/13; G16H 40/20

(52) U.S. Cl. CPC A61J 7/0076; A61J 1/06; A61K 35/76; B65G 1/1371; G16H 10/40; G16H 20/13; G16H 40/20; A61J 2200/70; A61J 2205/10; A61J 2205/60; B65G 61/00

(57) ABSTRACT

A phage dispensing method, phage dispensing apparatus a phage dispensing system configured to dispense a phage prescription. A dose calculation is performed using the patient weight, the target phage dose, a titer of a phage vial and a contaminant level of a phage vial to determine a dispensing set and associated dispensing volumes from phage vials stored in a phage storage apparatus. Storage locations and dose preparation instructions including respective dispensing volumes are provided to a pharmacist and a verification of the vials is performed to check the correct vials are removed from the phage storage apparatus.

### CROSS-REFERENCE TO RELATED APPLICATIONS

This application is a continuation of International Application No. PCT/US2022/076284, filed Sep. 12, 2022, which claims the benefit of U.S. Provisional Application No. 63/243,513, filed Sep. 13, 2021, and is a continuation-in-part of U.S. patent application Ser. No. 18/535,422, filed Dec. 11, 2023, which is a continuation of U.S. patent application Ser. No. 16/896,718, filed Jun. 9, 2020, now U.S. Pat. No. 11,911,345, issued Feb. 27, 2024, which is a National Stage Entry of International Application No. PCT/US2018/064645, filed Dec. 10, 2018, which claims the benefit of U.S. Provisional Application No. 62/597,008, filed Dec. 11, 2017, the entireties of which are incorporated herein by reference.

### TECHNICAL FIELD

The present disclosure relates to dispensing systems for pharmaceutical compositions. In a particular form the present disclosure relates to dispensing system for pharmaceutical compositions including Bacteriophages (“phages”).

### BACKGROUND

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Multiple drug resistant (MDR) bacteria are emerging at an alarming rate. Currently, it is estimated that at least 2 million infections are caused by MDR organisms every year in the United States leading to approximately 23,000 deaths. Further, the overuse of antibiotics as well as bacteria's natural evolution will likely lead to the generation of more virulent microorganisms. Genetic engineering and synthetic biology may also lead to the generation of additional highly virulent microorganisms.

For example, Staphylococcus aureus are gram positive bacteria that can cause skin and soft tissue infections (SSTI), pneumonia, necrotizing fasciitis, and blood stream infections. Methicillin resistant S. aureus (“MRSA”) is an MDR organism of great concern in the clinical setting as MRSA is responsible for over 80,000 invasive infections, close to 12,000 related deaths, and is the primary cause of hospital acquired infections. Additionally, the World Health Organization (WHO) has identified MRSA as organisms of international concern.

In view of the potential threat of rapidly occurring and spreading virulent microorganisms and antimicrobial resistance, alternative clinical treatments against bacterial infection are being developed. One such potential treatment for MDR infections involves the use of phage. Bacteriophages (“phages”) are a diverse set of viruses that replicate within and can kill specific bacterial hosts. The possibility of harnessing phages as an antibacterial was investigated following their initial isolation early in the 20th century, and they have been used clinically as antibacterial agents in some countries with some success. Notwithstanding, phage therapy was largely abandoned in the U.S. after the discovery of penicillin, and only recently has interest in phage therapeutics been renewed, particularly in light of the development Multiple Drug Resistant Infections (MDRI) such as due to antibiotic resistant bacteria.

Therapeutic use of phage typically comprises first identifying a phage strain likely to be efficacious against a bacteria associated with an infection (including a MDRI) of a specific patient. Phage are grown in batches at a manufacturing facility, and the purified phage is placed in a glass dispensing vial along with associated pharmaceutical ingredients such as excipients, carriers, buffers, and/or diluents. These may be stored in a low temperature (e.g. —80° C.) store at the dispensary. When provided with a phage prescription, a pharmacist removes a vial containing the desired phage from the low temperature store and must then extract an appropriate dose from the vial and combine with other pharmaceutical ingredients to manufacture a (patient ready) phage based treatment. The phage based treatment is then dispensed to the patient to treat the infection.

However, phage are quite different than most pharmaceutical compositions and creates a number of challenges in developing an efficient dispensing system. Most notably, there is significant batch to batch variability during manufacture of phage including concentration and contaminants, all of which the pharmacist must take into account when dispensing. Additionally the potency may change over time, and this will also vary based on batch and/or date of production, even for phage grown from the same batch, but subject to different processing and storage. Thus, unlike most pharmaceutical compositions stored at a dispensary that have uniform ingredients and potency (such that individual packages arc readily interchangeable), the individual phage vials available in a cold store containing the same phage but manufactured in different batches and/or at different times may have intrinsic variability which the pharmacist must take into account when dispensing. Phage are also expensive to manufacture, and thus storage and inventory at clinical sites must be efficiently managed to ensure sufficient supply. Phage Dispensing must thus take into account a range of patient, clinical, logistical and manufacturing factors. Additionally existing dispensing systems are often paper based or email based, and are typically cumbersome to use.

There is thus a need to develop a dispensing system which can manage these factors to ensure accurate and/or efficient dispensing at the point of care, or to at least to provide a useful alternative to existing dispensing systems.

### SUMMARY

According to a first aspect, there is provided a computer implemented method for dispensing a phage prescription, the method comprising:
receiving a phage prescription for a patient, the phage prescription comprising one or more phage to be dispensed, and determining a target phage dose range for each of the one or more phages to be dispensed;
receiving a patient weight and determining a maximum contaminant level for one or more contaminants based on the patient weight;
generating a dispensing set comprised of one or more phage vials stored in a phage storage apparatus by performing a dose calculation, comprising:
   identifying, for each phage in the phage prescription, at least one candidate phage vials in the phage storage apparatus that contain the respective phage and determining a dispensing volume for one or more candidate phage vials based on a titer of the respective candidate phage vial such that the total dispensing volume from combining the dispensing volume for the one or more candidate phage vials will contain an amount of phage within the target phage dose range for the respective phage and an amount of contaminant, for each contaminant in the one or more contaminants, that is below the maximum contaminant level for the respective contaminant;
providing a storage location and dose preparation instructions for each of the one or more candidate phage vials in the dispensing set to a user, wherein the dose preparation instructions comprises the associated dispensing volume for each of the phage vials in the dispensing set; and
receiving a phage vial identifier (ID) obtained from a phage vial and checking the phage vial ID matches a stored phage vial ID of one of the phage vials in the dispensing set, and if the phage vial ID matches then updating an electronic phage inventory to record the dispensing of the phage vial otherwise issuing a warning to the user.

In one form, the phage prescription comprises a target phage dose for each of the one or more phages to be dispensed and the target phage dose range for each of the one or more phages to be dispensed is determined from the respective target phage dose.

In a further form, a set of predefined non-overlapping target phage dose ranges are stored, and determining the target phage dose comprises determining which target phage dose range in the set contains the target phage dose.

In one form, a plurality of dose range labels and associated dose ranges are stored, and the phage prescription includes the dose range label and determining the target phage dose range comprises looking up the dose range associated with the dose range label.

In one form, the target phage dose is an upper limit of target phage dose range.

In one form, determining a dispensing volume for one or more candidate phage vials comprises determining a dispensing volume for a first candidate phage vial using the titer of the first candidate phage vial, and if the dispensing volume is less than the volume required to dispense the phage prescription for the respective phage, then searching for one or more additional candidate phage vials which are identical phage vials to the first candidate phage vial and if one or more identical phage vials to the first phage candidate phage vial are located, then adding one or more of the one or more identical phage vials to the candidate set and storing an associated dispensing volume for each of the one or more identical phage vials if the amount of contaminant, for each contaminant in the one or more contaminants, is below the maximum contaminant level for the respective contaminant, and if the dispensing set does not comprise sufficient candidate phage vials and associated dispensing volumes to enable dispensing of the phage prescription for the respective phage, then repeating until the dispensing set comprises sufficient candidate phage vials to dispense the phage prescription for the respective phage such that the amount of each contaminant is below the maximum contaminant level for the respective contaminant.

In a further form, two vials are identical if their titer and contaminant levels are within a predefined range of each other, and/or two vials are identical if they have the same lot number.

In a further form, the method further comprises determining a target phage dose for each of the one or more phages to be dispensed and using an optimization method to search for a combination of candidate phage vials and associated dispensing volumes which when combined generate a total dose closest to the target dose subject to the constraint that the amount of contaminant, for each contaminant in the one or more contaminants, is below the maximum contaminant level for the respective contaminant.

In a further form, the one or more contaminants comprising an endotoxin level, and the associated maximum contaminant level is weight (in kg)×5 EU/kg.

In one form, a computing apparatus is operatively connected to the phage storage apparatus, the phage vial identifier (ID) is a barcode or a radio frequency identification (RFID) tag, and the computing apparatus comprises;
at least one processor,
at least one memory;
at least one display device;
one or more user interface devices; and
a reader apparatus configured to read a barcode and/or a RFID tag,
wherein the one or more user interface devices is configured to request a user input the patient weight, and on receiving the patient weight the computing apparatus generates the dispensing set and displays the storage location and dose preparation instructions for each of the candidate phage vials in the dispensing set on the display device and obtaining the phage vial identifier comprises reading the barcode or the RFID tag of a vial using the reader apparatus.

In one form, providing a storage location and dose preparation instructions further comprises printing and/or emailing the storage location and dose preparation instructions to the user.

In one form, the computing apparatus is a portable computing apparatus and the display device is a touch screen device which also acts as a user interface device.

In one form, the computing apparatus displays an electronic certificate of analysis for each of the phage vials in the dispensing set.

In one form, the electronic phage inventory comprises a phage record for each of the plurality of vials stored in the phage storage apparatus, and each phage record stores a phage location, a phage titer, at least one contaminant level, a phage vial ID and an electronic certificate of analysis for the respective phage vial.

In one form, after updating the electronic phage inventory, a request is sent to a remote store to ship additional phage vials to the phage storage apparatus.

According to a second aspect, there is provided a phage dispensing apparatus comprising:
at least one memory, and
at least one processor wherein the memory comprises instructions to configure the processor to:
receive a phage prescription for a patient, the phage prescription comprising one or more phage to be dispensed, and determine a target phage dose range for each of the one or more phages to be dispensed;
receive a patient weight and determining a maximum contaminant level for one or more contaminants based on the patient weight;
generate a dispensing set comprised of one or more phage vials stored in a phage storage apparatus by performing a dose calculation, comprising:
   identifying, for each phage in the phage prescription, at least one candidate phage vials in the phage storage apparatus that contain the respective phage and determining a dispensing volume for one or more candidate phage vials based on a titer of the respective candidate phage vial such that the total dispensing volume from combining the dispensing volume for the one or more candidate phage vials will contain an amount of phage within the target phage dose range for the respective phage and an amount of contaminant, for each contaminant in the one or more contaminants, that is below the maximum contaminant level for the respective contaminant;
provide a storage location and dose preparation instructions for each of the one or more candidate phage vials in the dispensing set to a user, wherein the dose preparation instructions comprises the dispensing volume for each of the phage vials in the dispensing set; and
receive a phage vial identifier (ID) obtained from a phage vial and checking the phage vial ID matches a stored phage vial ID of one of the phage vials in the dispensing set, and if the phage vial ID matches then updating an electronic phage inventory to record the dispensing of the phage vial otherwise issuing a warning to the user.

In one form, the phage prescription comprises a target phage dose for each of the one or more phages to be dispensed and the target phage dose range for each of the one or more phages to be dispensed is determined from the respective target phage dose.

In a further form, a set of predefined non-overlapping target phage dose ranges are stored, and determining the target phage dose comprises determining which target phage dose range in the set contains the target phage dose.

In one form, a plurality of dose range labels and associated dose ranges are stored, and the phage prescription includes the dose range label and determining the target phage dose range comprises looking up the dose range associated with the dose range label.

In one form, the target phage dose is an upper limit of target phage dose range.

In one form, determining a dispensing volume for one or more candidate phage vials comprises determining a dispensing volume for a first candidate phage vial using the titer of the first candidate phage vial, and if the dispensing volume is less than the volume required to dispense the phage prescription for the respective phage, then searching for one or more additional candidate phage vials which are identical phage vials to the first candidate phage vial and if one or more identical phage vials to the first phage candidate phage vial are located, then adding one or more of the one or more identical phage vials to the candidate set and storing an associated dispensing volume for each of the one or more identical phage vials if the amount of contaminant, for each contaminant in the one or more contaminants, is below the maximum contaminant level for the respective contaminant, and if the dispensing set does not comprise sufficient candidate phage vials and associated dispensing volumes to enable dispensing of the phage prescription for the respective phage, then repeating until the dispensing set comprises sufficient candidate phage vials to dispense the phage prescription for the respective phage such that the amount of each contaminant is below the maximum contaminant level for the respective contaminant.

In a further form, two vials are identical if their titer and contaminant levels are within a predefined range of each other, and/or two vials are identical if they have the same lot number.

In a further form, the method further comprises determining a target phage dose for each of the one or more phages to be dispensed and using an optimization method to search for a combination of candidate phage vials and associated dispensing volumes which when combined generate a total dose closest to the target dose subject to the constraint that the amount of contaminant, for each contaminant in the one or more contaminants, is below the maximum contaminant level for the respective contaminant.

In a further form, the one or more contaminants comprising an endotoxin level, and the associated maximum contaminant level is weight (in kg)×5 EU/kg.

In one form, the phage dispensing apparatus is operatively connected to the phage storage apparatus, the phage vial identifier (ID) is a barcode or a radio frequency identification (RFID) tag, and the phage dispensing apparatus further comprises;
at least one display device;
one or more user interface devices; and
a reader apparatus configured to read a barcode and/or a RFID tag,
wherein the one or more user interface devices is configured to request a user input the patient weight, and on receiving the patient weight the computing apparatus generates the dispensing set and displays the storage location and dose preparation instructions for each of the candidate phage vials in the dispensing set on the display device, and obtaining the phage vial identifier comprises reading the barcode or RFID tag of a vial using the reader apparatus.

In one form, providing a storage location and dose preparation instructions further comprises printing and/or emailing the storage location and dose preparation instructions to the user.

In one form, the phage dispensing apparatus is a portable computing apparatus and the display device is a touch screen device which also acts as a user interface device.

In one form, the phage dispensing apparatus displays an electronic certificate of analysis for each of the phage vials in the dispensing set.

In one form, the electronic phage inventory comprises a phage record for each of the plurality of vials stored in the phage storage apparatus, and each phage record stores a phage location, a phage titer, at least one contaminant level, a phage vial ID and an electronic certificate of analysis for the respective phage vial.

In one form, after updating the electronic phage inventory, a request is sent to a remote store to ship additional phage vials to the phage storage apparatus.

According to a third aspect, there is provided a non-transitory, computer program product comprising computer executable instructions for dispensing a phage prescription, the instructions executable by a computer to:
receive a phage prescription for a patient, the phage prescription comprising one or more phage to be dispensed, and determine a target phage dose range for each of the one or more phages to be dispensed;
receive a patient weight and determining a maximum contaminant level for one or more contaminants based on the patient weight;
generate a dispensing set comprised of one or more phage vials stored in a phage storage apparatus by performing a dose calculation, comprising:
   identifying, for each phage in the phage prescription, at least one candidate phage vial in the phage storage apparatus that contain the respective phage and determining a dispensing volume for one or more candidate phage vials based on a titer of the respective candidate phage vial such that the total dispensing volume from combining the dispensing volume for the one or more candidate phage vials will contain an amount of phage within the target phage dose range for the respective phage and an amount of contaminant, for each contaminant in the one or more contaminants, that is below the maximum contaminant level for the respective contaminant;
providing a storage location and dose preparation instructions for each of the one or more candidate phage vials in the dispensing set to a user, wherein the dose preparation instructions comprises the dispensing volume for each of the phage vials in the dispensing set; and
receiving a phage vial identifier (ID) obtained from a phage vial and checking the phage vial ID matches a stored phage vial ID of one of the phage vials in the dispensing set, and if the phage vial ID matches then updating an electronic phage inventory to record the dispensing of the phage vial otherwise issuing a warning to the user.

According to a fourth aspect, there is provided a phage dispensing system comprising:
a phage management apparatus comprising:
at least one memory, and
at least one processor wherein the memory comprises instructions to configure the processor to implement an electronic phage inventory, wherein the electronic phage inventory comprise a phage record for each of a plurality of vials, and each phage record stores a phage location, a phage titer, at least one contaminant level, a phage vial ID and an electronic certificate of analysis for the respective phage vial; and
a plurality of phage dispensing apparatus which in use are located at a plurality of clinical sites, each phage dispensing apparatus comprising:
a phage storage apparatus that stores a plurality of phage vials; and a phage dispensing apparatus comprising at least one memory, and at least one processor wherein each phage dispensing apparatus is configured to:
receive a phage prescription for a patient, the phage prescription comprising one or more phage to be dispensed, and determine a target phage dose range for each of the one or more phages to be dispensed;
receive a patient weight and determine a maximum contaminant level for one or more contaminants based on the patient weight;
generate a dispensing set comprised of one or more phage vials stored in a phage storage apparatus by performing a dose calculation, comprising:
   identifying, for each phage in the phage prescription, at least one candidate phage vials in the phage storage apparatus that contain a phage and determining a dispensing volume for one or more candidate phage vials based on a titer of the respective candidate phage vial such that the total dispensing volume from combining the dispensing volume for the one or more candidate phage vials will contain an amount of phage within the target phage dose range for the respective phage and an amount of contaminant, for each contaminant in the one or more contaminants, that is below the maximum contaminant level for the respective contaminant;
provide a storage location and dose preparation instructions for each of the one or more candidate phage vials in the dispensing set to a user, wherein the dose preparation instructions comprise the dispensing volume for each of the respective phage vials in the dispensing set; and
receive a phage vial identifier (ID) obtained from a phage vial and checking the phage vial ID matches a stored phage vial ID of one of the phage vials in the dispensing set, and if the phage vial ID matches then sending an update to the electronic phage inventory to record the dispensing of the phage vial otherwise issuing a warning to the user.

In one form, the phage management apparatus is further configured to communicate with each of the plurality of phage dispensing apparatus to store a record of each dispensed phage prescription.

In one form, the phage management apparatus is further configured to monitoring the dispensed phage and generate manufacturing orders to a phage manufacturer and/or movement requests between phage storage sites.

In one form, the phage management apparatus is further configured to communicate with a phage testing laboratory to receive phage susceptibility test results for a patient sample, and sends a treatment recommendation to a treating clinician including a proposed phage prescription.

### BRIEF DESCRIPTION OF DRAWINGS

Embodiments of the present disclosure will be discussed with reference to the accompanying drawings wherein:

FIG. 1A is a schematic diagram of an embodiment of a phage dispensing system;

FIG. 1B is a flow chart of a method for dispensing a phage prescription according to an embodiment;

FIG. 2A is a flowchart of a dispensing method 200 according to an embodiment;

FIG. 2B is a flowchart of a dose calculation according to another embodiment;

FIG. 3 is a schematic diagram of a computing apparatus according to an embodiment; and

FIG. 4 is a representation of a certificate of analysis of a phage vial according to an embodiment.

In the following description, like reference characters designate like or corresponding parts throughout the figures.

### DESCRIPTION OF EMBODIMENTS

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules. “A phage formulation” can mean at least one phage formulation, as well as a plurality of phage formulations, i.e., more than one phage formulation. As understood by one of skill in the art, the term “phage” can be used to refer to a single phage or more than one phage.

The present invention can “comprise” (open ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

As used herein, a “subject” is a vertebrate, preferably a mammal, more preferably a human. Mammals include, but are not limited to, murines, simians, humans, farm animals, sport animals, and pets. In other preferred embodiments, the “subject” is a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutan, gibbon). In other embodiments, non-human mammals, especially mammals that are conventionally used as models for demonstrating therapeutic efficacy in humans (e.g., murine, primate, porcine, canine, or rabbit animals) may be employed. Preferably, a “subject” encompasses any organisms, e.g., any animal or human, that may be suffering from a bacterial infection, particularly an infection caused by a multiple drug resistant bacterium.

As understood herein, a “subject in need thereof” includes any human or animal suffering from a bacterial infection, including but not limited to a multiple drug resistant bacterial infection, a microbial infection or a polymicrobial infection. Indeed, while it is contemplated herein that the methods may be used to target a specific pathogenic species, the method can also be used against essentially all human and/or animal bacterial pathogens, including but not limited to multiple drug resistant bacterial pathogens. Thus, in a particular embodiment, by employing the methods of the present invention, one of skill in the art can design and create personalized phage formulations against many different clinically relevant bacterial pathogens, including multiple drug resistant (MDR) bacterial pathogens.

As understood herein, an “effective amount” of a pharmaceutical composition refers to an amount of the composition suitable to elicit a therapeutically beneficial response in the subject, e.g., eradicating a bacterial pathogen in the subject. Such response may include e.g., preventing, ameliorating, treating, inhibiting, and/or reducing one of more pathological conditions associated with a bacterial infection.

The term “dose” or “dosage” as used herein refers to physically discrete units suitable for administration to a subject, each dosage containing a predetermined quantity of the active pharmaceutical ingredient calculated to produce a desired response.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term “about” means within an acceptable error range for the particular value, such as ±1-20%, preferably ±1-10% and more preferably ±1-5%. In even further embodiments, “about” should be understood to mean+/−5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition is described as containing characteristics A, B, and/or C, the composition can contain A feature alone; B alone; C alone; A and B in combination; A and C in combination; B and C in combination; or A, B, and C in combination.

The term “phage sensitive” or “sensitivity profile” means a bacterial strain that is sensitive to infection and/or killing by phage and/or in growth inhibition. That is phage is efficacious or effective in inhibiting growth of the bacterial strain.

The term “phage insensitive” or “phage resistant” or “phage resistance” or “resistant profile” is understood to mean a bacterial strain that is insensitive, and preferably highly insensitive to infection and/or killing by phage and/or growth inhibition. That is phage is not efficacious or is ineffective in inhibiting growth of the bacterial strain.

The term “intermediate phage sensitive” is understood to mean a bacterial strain that exhibits a sensitivity to infection by phage that is in between the sensitivity of phage sensitive and phage insensitive strains.

As used herein, “predictive patterns” are genomic patterns identified in the plurality of bacterial strains and/or in the plurality of phage strains that are associated correlate with a “sensitivity profile”, “resistant profile”, or “intermediate sensitivity profile” of a bacterium.

As used herein, a “phage-host specificity profile” is used interchangeably with a “phage-host sensitivity profile” and a “phage susceptibility test” for a bacterial isolate, and comprises data relating to a bacterium's sensitivity or resistance to a plurality of different phage. The phage-host specificity profile or phage susceptibility of a bacterial isolate can be experimentally derived, computationally predicted, or some combination.

A “phage treatment”, “therapeutic phage formulation”, “therapeutically effective phage formulation”, “phage formulation” or like terms as used herein are understood to refer to a composition comprising one or more phage which can provide a clinically beneficial treatment for a bacterial infection when administered to a subject in need thereof. A “therapeutic phage cocktail”, “therapeutically effective phage cocktail”, “phage cocktail” or like terms as used herein are understood to refer to a composition comprising a plurality of phage which can provide a clinically beneficial treatment for a bacterial infection when administered to a subject in need thereof. Preferably, therapeutically effective phage cocktails are capable of infecting the infective parent bacterial strain as well as the emerging resistant bacterial strains that may grow out after elimination of the parent bacterial strain.

As used herein, the term “composition” encompasses “phage based treatments”, “phage formulations” and the like which include, but are not limited to, pharmaceutical compositions comprising one or more purified phage. “Pharmaceutical compositions” are familiar to one of skill in the art and typically comprise active pharmaceutical ingredients formulated in combination with inactive ingredients selected from a variety of conventional pharmaceutically acceptable excipients, carriers, buffers, and/or diluents. The term “pharmaceutically acceptable” is used to refer to a non-toxic material that is compatible with a biological system such as a cell, cell culture, tissue, or organism (or at least non-toxic in amounts typically used). Examples of pharmaceutically acceptable excipients, carriers, buffers, and/or diluents are familiar to one of skill in the art and can be found, e.g., in Remington's Pharmaceutical Sciences (latest edition), Mack Publishing Company, Easton, Pa. For example, pharmaceutically acceptable excipients include, but are not limited to, wetting or emulsifying agents, pH buffering substances, binders, stabilizers, preservatives, bulking agents, adsorbents, disinfectants, detergents, sugar alcohols, gelling or viscosity enhancing additives, flavoring agents, and colors. Pharmaceutically acceptable carriers include macromolecules such as proteins, polysaccharides, polylactic acids, polyglycolic acids, polymeric amino acids, amino acid copolymers, trehalose, lipid aggregates (such as oil droplets or liposomes), and inactive virus particles. Pharmaceutically acceptable diluents include, but are not limited to, water, saline, and glycerol.

As used herein, the term “purified” refers to a preparation that is substantially free of unwanted substances in the composition, including, but not limited to biological materials e.g., toxins, such as for example, endotoxins, nucleic acids, proteins, carbohydrates, lipids, or subcellular organelles, and/or other impurities, e.g., metals or other trace elements, that might interfere with the effectiveness of the cocktail. As used herein, terms like “high titer and high purity”, and “very high titer and very high purity” refers to degrees of purity and titer that are familiar to one of skill in the art.

As used herein, the term “vial” encompasses a range of containers which can be used to contain or store an individual phage in solution or a phage cocktail and include lids. Vials may be single dose or multi dose containers. Single dose containers may be sealed including ampules and other hermetically sealed vials. Multi dose containers may store phage at a specific concentration or comprise a partitioned interior. Vials may be made of suitable glasses, plastics, composites or metal, that can store the phage in a viable state for an extended period of time, including at low temperatures such as −80° C.

Bacteria to be treated using the phage based treatment and compositions described herein include any bacterial pathogen that poses a health threat to a subject. These bacterial include, but are not limited to the “ESKAPE” pathogens (Enterococcus faecium, Staphylococcus aureus, Klebsiella pneumonia, Acinetobacter baumannii, Pseudomonas aeruginosa, and Enterobacter sp), which are often nosocomial in nature and can cause severe local and systemic infections. Among the ESKAPE pathogens, A. baumannii is a Gram-negative, capsulated, opportunistic pathogen that is easily spread in hospital intensive care units. Many A. baumannii clinical isolates are also MDR, which severely restricts the available treatment options, with untreatable infections in traumatic wounds often resulting in prolonged healing times, the need for extensive surgical debridement, and in some cases the further or complete amputation of limbs. Further preferred bacteria strains include G. mellonella.

One of skill in the art will appreciate that bacteria subject to the method described herein include, but are not limited to, multidrug resistant bacterial strains. As understood herein, the terms, “multidrug resistant”, “multiple drug resistant”, “multiple drug resistance” (MDR) and like terms may be used interchangeably herein, and are familiar to one of skill in the art, i.e., a multiple drug resistant bacterium is an organism that demonstrates resistance to multiple antibacterial drugs, e.g., antibiotics.

In preferred embodiments, examples of MDR bacteria are methicillin resistant S. aureus (MRSA) and vancomycin-resistant Enterococci (VRE).

As understood herein, the term “diverse sources” includes a wide variety of different places where phage may be found in the environment including, but not limited to, any place where bacteria are likely to thrive. In fact, phage are universally abundant in the environment, making the isolation of new phage very straightforward. The primary factors affecting the successful isolation of such phage are the availability of a robust collection of clinically relevant bacterial pathogens to serve as hosts, and access to diverse environmental sampling sites.

As used herein, the term “determining” encompasses a wide variety of actions. For example, “determining” may include calculating, computing, processing, estimating, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

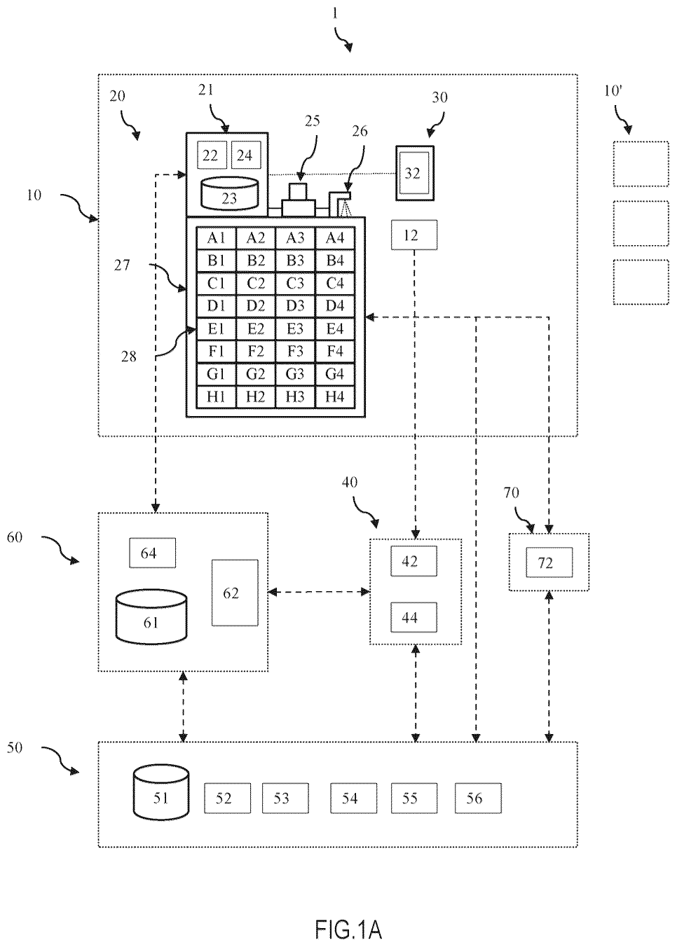

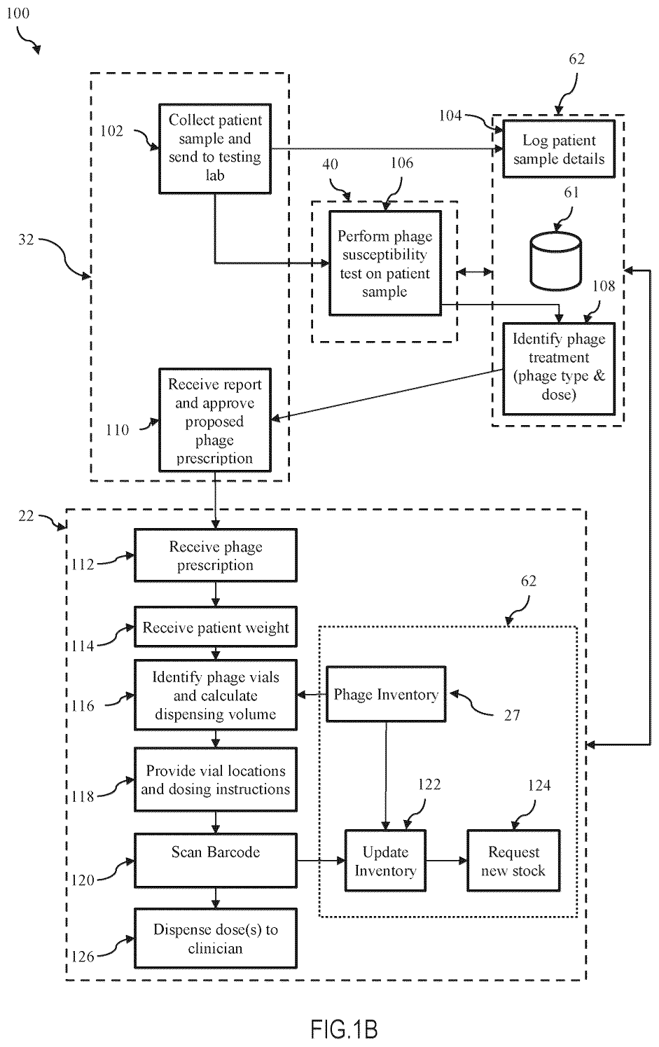

Embodiments of computer implemented methods for dispensing a phage prescription and associated phage dispensing systems will now be described with reference to FIGS. 1A and 1B. FIG. 1A is a schematic diagram of an embodiment of a phage dispensing system 1 and FIG. 1B is a flow chart of a method 100 for dispensing a phage prescription according to an embodiment.

As shown in FIG. 1A a phage dispensing apparatus 20 is located at a clinical site 10. There may be multiple clinical sites (10′) and each clinical site may have one or more phage dispensing apparatus 20. The system further includes one or more testing laboratories 40 that perform phage susceptibility tests, one or more a manufacturing sites 50, a phage management apparatus 60, and one or more phage storage sites 70. A testing laboratory 40, the phage management system 60, and a storage site may all be hosted at a phage manufacturing site 50.

The phage dispensing apparatus 20 comprises a computing apparatus 21, such as a desktop computer or mobile computing device (e.g., tablet, laptop) and a phage storage apparatus 27 such as a low temperature (−80 C) refrigerator used to store phage vials to be used in preparing phage prescriptions to produce phage based treatments for dispensing to patients 12. The computing apparatus 21 comprises at least one memory, at least one processor 24 and one or more user interface devices, and is configured to execute a phage dispensing application (i.e., software) 22 which provides a user interface to guide dispensing of a phage prescription by a pharmacist. The one or more user interface devices may include one or more display devices, such as a LCD screen or a touchscreen, and a reader apparatus 26 configured to read a phage vial identifier (ID) in the form of a barcode and/or an RFID tag located on or adhered to the vial (e.g. in the form of a label with an adhesive backing). The reader apparatus 26 may be a barcode scanner with an optical source and optical receiver, or camera used to capture an image containing the barcode which is then processed (scanned) to extract or recognize the barcode in the image. Alternatively or additionally the reader apparatus may be configured to read a RFID tag on the vial. The reader apparatus may comprise an antenna which transmits an interrogator signal and a receiver which receives a reply signal (a message) encoding the phage vial identifier from an RFID tag. The reader apparatus is configured to process the received signal to extract (e.g. decode) the phage vial identifier from the reply signal. The RFID tag may store additional information, such as lot number, date of manufacture, etc., which may also be encoded in the reply signal, and which is also extracted from the replay signal. The one or more user interface devices may be integral or incorporated in the computer apparatus 21 such as a touchscreen, camera and microphone, or they may be peripheral input/output devices such as a printer 25 and reader apparatus (e.g. barcode scanner or RFID reader) 26 which are operatively connected to the computing apparatus 21 over a wired or wireless connection. The phage dispensing software 22 may also communicate with a dispensing application 32 executing on a computing device 30 such as a smartphone, tablet computer or desktop computer of a clinician or pharmacist. For example, a clinician may prepare a phage prescription using the dispensing application 32 which is sent to the phage dispensing software 22 for dispensing by a pharmacist. The phage dispensing software 22 may also communicate with a phage management application 62 as part of a remotely located phage management system 60. In one embodiment the phage management system 62 is further configured to interface or communicate with a Laboratory Information Management System (LIMS) associated with the treatment site (e.g. the hospital) and/or an electronic health record (EHR) system to record the dispensing of the prescription to the patient (in their EHR record). Communication with LIMS or EHR systems may be used to lookup data stored in such systems or to record dispensing actions such as to update a patient record.

The phage storage apparatus 27 is divided into a plurality of storage locations 28. These may be arranged as a regular array of boxes to allow each box to be assigned a two dimensional coordinate such as (row, column), e.g. FIG. 1A shows 32 bays arranged as Rows A to H and columns 1 to 4. Each box may be further divided into a series of bays within the box to give a third dimension to the location (coordinate). In one embodiment each bay stores an individual phage vial thus allowing each phage vial to be assigned a unique location within the phage storage apparatus according to a three dimensional coordinate e.g. (Row, Column, Bay). In other embodiments multiple phage vials may be placed within the same box or bay, in which case the location may be shared with other phage vials.

The phage manufacturing site 50 performs manufacture (production) of phage using manufacturing equipment 53 such as bioreactors used to grow phage in batches. Purified phage is extracted and combined with associated pharmaceutical ingredients such as excipients, carriers, buffers, and/or diluents and sealed into phage vials. Phage vials may contain a single phage, or multiple phage. Each phage vial is thus a phage formulation or composition comprising one or more purified phage. The phage manufacturing site may additionally include quality control (or quality assurance) laboratories 54, phage hunting/identification facilities 54 and genomics laboratories, for example which identify genomic patterns in bacteria or phage which may be used to determine sensitivity or resistance profiles, or these functions may be outsourced to other sites. The site may also include a process control and management system 52 with an associated memory 51 such as a database, which is used to control, monitor, and log the production and quality control activities performed at the phage manufacturing site.

The phage management system 60 comprises one or more computing apparatus including one or more memories 61 and one or more processors 64 and is configured to execute the phage management application 62. The phage management system may be located at a manufacturing site 50, at a clinical site 10, including as part of phage dispensing apparatus, or at a separate location such as a data center or hosted in a cloud computing environment. The phage management application may include one or more databases and is used to store and maintain a record of the phage manufactured, stored and dispensed in the system 1. The phage management application 62 may include an electronic phage inventory which stores a phage record for each vial that including tracking data, manufacturing data, pharmaceutical data and QC/QA data. This may include the current storage location of the phage vial, manufacturing lot number, date of manufacture, and test results such as phage titer, pH, and levels of contaminants such as an endotoxin level. The metadata for a phage vial may include an electronic certificate of analysis generated by the quality control laboratory 54 or a link to a remotely stored electronic certificate of analysis. The phage management application 62 may communicate with the process control and management system 52 at each manufacturing site for example to obtain manufacturing data and quality control data on phage manufactured by the site. The phage management application 62 may maintain a count of the number of vials available for each phage strain, their locations, the age of vials (and expiry dates), and relevant pharmaceutical properties, such as contained in a certificate of analysis.

After manufacture, phage is distributed (shipped) to phage storage apparatus 27 at clinical sites 10 and or other storage sites 70 such as pharmacies, regional hospitals and/or warehouses. Each storage site 70 may execute a local phage management application 72 which maintains a record of locally stored phage and which may communicate with the phage management application 62 (and the electronic phage inventory). The phage management application 62 may be used to monitor phage stocks at each storage site (including expiry date of stored phage), and send manufacturing orders for specific phage batches to a manufacturing site and/or movement requests (shipping orders) to resupply a phage storage site, and/or move (redistribute) phage stocks between different storage locations. The phage management application 72 may also perform data analytics and provide input for phage hunting, including orders to send samples from the phage testing laboratory to the manufacturing site to assist in phage hunting.

The testing laboratory 40 is used to perform phage susceptibility tests 44 on samples obtained from patients to identify one or more phage likely to be efficacious against the patient's infection (or more specifically bacteria associated with the patient's infection). The testing laboratory may be located at a clinic 10, an independent site, or as part of the manufacturing facility 50. A range of phage susceptibility tests may be performed which measure bactericidal activity of phage against the bacteria in the patient sample, or a bacterial isolate obtained from the patient sample. These include phage that can generate clear point plaques on the bacterial sample, phage that demonstrate lytic characteristics using a rapid streak method on a plate, bacterial lysis of through assessment of absorbance difference in turbidity with small or large batch assays, or delay in bacterial growth. Typically, the testing will be performed in parallel, testing many (e.g., hundreds or thousands) of phage against the bacterial isolate to identify which phage strains are likely to be efficacious against the patient's infection. In one embodiment the phage susceptibility test is a plaque assay and/or a host-phage response assay (using the bacterial isolate as the host) including a colorimetric assay that measures bacterial respiration as a proxy for bacterial growth performed using an automated OmniLog™ system. A laboratory management application 42 records details of the patient sample and the phage susceptibility tests and generates associated reports containing the test results and recommendations. These reports may also take into account geographic information such as the presence of similar infections at the patient site, which may indicate a common bacterial strain infecting patient's at the site, as well as genomic and bioinformatics analysis of the bacterial isolate to identify possible efficacious phage.

Turning now to FIG. 1B, there is shown a flow chart of a method 100 for dispensing a phage prescription according to an embodiment.

As a precursor to the dispensing a phage prescription is first generated by a treating clinician. For example, a treating clinician may identify that a patient 12 may benefit from a phage based treatment and proceed to collect a patient sample which is sent to a phage susceptibility laboratory for testing 102. In one embodiment the clinician may access a user interface of the dispensing application 32 to request a phage susceptibility test 44 of a patient sample by the phage laboratory 40. The user interface is configured to collect a patient identifier as well as any other relevant information regarding the patient and their condition, as well as logistical details such as patient sample location or where they are to be treated. The user interface may also be configured to interact with other computing systems such as patient records systems or other clinical system to assist in collection of any required information. The information may also be sent to the phage management application 62 to log patient sample details 104. The patient sample details logged by the phage management application may include date of the date of the sample collection, bacterial identifier (ID), a geographic location of the patient (or clinic), clinical indications, Antimicrobial or Antibiotic Susceptibility Testing (AST) data, patient data and historical outcomes of other patients.

In one embodiment the phage management application determines the most suitable phage susceptibility laboratory 40 to send the sample to, and coordinates transportation of the patient sample to the phage susceptibility laboratory 40 and subsequent phage susceptibility testing. Once the patient sample arrives at the phage susceptibility laboratory 40, a phage susceptibility test is performed on the patient sample 106. The results are provided to the phage management application 62 and used to identify a potential phage treatment such as one or more phage types (or strains) and respective doses 108. The patient record may be updated with the phage susceptibility test results. Once a potential phage treatment is identified a report is sent to the clinician including a treatment recommendation that includes a proposed phage prescription (or treatment) 110. This may include the suggested phage type (or strain) and target dose level (for each phage type. The target dose level (PFU) may be used to determine a dispensing volume (mL) from a vial using the titer (PFU/mL) of the vial such as by dividing the titer by the target dose level. The target dose level may be a specific value, a target dose range, or both a target dose level and an allowable target dose range, i.e. the dose should be as close as possible to the target dose level, but any dose within the target dose range is regarded as acceptable. The clinician may then approve the phage prescription and provide to the pharmacist or dispensing apparatus for subsequent dispensing.

Phage susceptibility tests typically take many hours (10-24 or more). Thus, in one embodiment the phage management application 62 may make an initial therapy available to enable treatment to begin immediately upon receipt of patient sample details 104. This may be based on details such as patient location, symptoms and historical data such as similar infections at the same site which were successfully treated using a phage composition. For example, a cluster of similar cases at the patient site may suggest the patient has contracted the same infection as previous patients, and thus a previously successful phage treatment may be recommended to the clinician as an early intervention (from which the clinician can prepare a phage prescription).

The phage dispensing application 22 (executing on a computing apparatus operatively connected to a phage dispensing apparatus 20) then receives a phage prescription for the patient 12. The phage prescription may comprise one or more phage to be dispensed, and one or both of a target phage dose or target phage dose range, for each of the one or more phages to be dispensed (i.e., the regiment description), along with other metadata such as patient name or identifier, treating clinician, time and date, location, etc. This may be electronically sent to the phage dispensing application 22 by the dispensing application 32 used by the clinician, or, upon electronic approval of the proposed phage prescription by the clinician 110 (for example via a dispensing application 32), the system may send the approved phage prescription to the phage dispensing application 22. Alternatively, a physical or electronic prescription may be provided to a pharmacist who manually enters details of the phage prescription into the phage dispensing application 22.

The target phage dose range comprises a lower range limit and an upper range limit. The lower range limit and upper range limits will typically be inclusive or one will be inclusive and the other exclusive. For example the lower limit may be inclusive and the upper limit may be exclusive so that the range extends up to, but not including the upper range limit (e.g. lower limit≤range<upper limit) or vice versa. In one embodiment both lower range limit and upper range limits are exclusive limits.

In one embodiment the phage prescription defines a target dose range (e.g. 1e9-3c9 PFU) or a label for a predefined target dose range (e.g. low=1e9-3e9 PFU; high=1e10-3e10 PFU). The label and the associated target dose range may be stored by the phage dispensing apparatus or another storage device accessible by the phage dispensing application. In this case determining the target phage dose range comprises mapping the label to the predefined range (e.g. looking up the target dose range using the label as the search key). If a target phage dose is not specified, a target phage dose (within the range) may be defined according to a predefined rule, such as the upper limit of the provided dose range, the midpoint of the provided dose range or the lower limit of the provided dose range.

In one embodiment the phage prescription includes a target phage dose for a phage, which is then used to determine the target phage dose range. For example the target phage dose may be a specific number (e.g. 2e9 PFU) and the dose range may be determined based on a predetermined amount (e.g. ±le9 PFU) or percentage (e.g. ±5%). The amounts or percentages may be based on a log scale. Additionally the target dose value need not be the mid-point of the range. That is a lower interval, defined as the (absolute) difference between a lower range limit to the target dose value, may be different from an upper interval defined as the (absolute) difference between the target dose value and upper range limit (i.e. uneven intervals). In some embodiments the phage dispensing apparatus may store a predefined set of non-overlapping target phage dose ranges (i.e. multiple ranges), and determining the target dose range may include determining which one of the multiple dose ranges the target dose range falls within. The set of non-overlapping target phage dose ranges may be contiguous ranges where an upper limit of a first range is exclusionary (i.e. the range is up to the upper range limit) and the lower range limit of the next range is the same value as the upper range limit of the previous range, but is inclusive of the lower range limit (or vice versa where a lower range limit is exclusionary and the previous upper range limit is inclusive). In one embodiment if the target dose range does not fall within any of the multiple dose ranges a warning may be issued requesting a manual dose range to be entered. Alternatively a nearest dose range may be determined, such as a closest dose range with an upper range limit below the target dose range, or the closest dose range within a predefined percentage (e.g. 5%). In one embodiment the user interface may present the target phage dose range to the clinician (or pharmacist) or request the clinician (or pharmacist) to confirm the target phage dose range. In one embodiment the user interface may allow the clinician to enter the target dose range (and log the entry).

The phage dispensing application 22 is also configured to receive the patient's weight 114. In one embodiment the user interface of the phage dispensing application 22 prompts the pharmacist for the patient's weight. In one embodiment further processing of the dispensing is paused until the patient's weight is received. Sanity checks may also be performed to ensure the weight is within a typical range to prevent data errors (and the pharmacist may be prompted to recheck the weight in cases of large variation from a previous weight or typical range). In another embodiment, a patient weight may be provided by the clinician as part of metadata associated with the prescription, or obtained from a recent measurement such as a measurement stored in an electronic health record for the patient, and the clinician is asked to verify (or confirm) the use of the weight measurement. Metadata regarding the measurement, such as the person who made the measurement, the location where the measurement was made, and the time of the measurement may also be provided. The valid period for the recent measurement may be within the previous 12, 24, 36, 48, 72 hours or even 168 hours. If the pharmacist does not consider the measurement is sufficiently recent, they can re-measure and reenter a new measurement. The phage dispensing application 22 uses the patient's weight to determine a maximum contaminant level for one or more contaminants that may be present in the vials. In one embodiment the one or more contaminants includes an endotoxin (EU) level and the maximum endotoxin level (Max EU) is calculated as weight (kg)*5 EU/kg.

The phage dispensing application 22 then proceeds to generate a dispensing set comprised of one or more phage vials stored in the phage storage apparatus 27 by performing a dose calculation 116. The dose calculation determines an associated dispensing volume for each phage vial in the dispensing set such that the total dispensing volume contains an amount of phage within the target phage dose range for the respective phage. This comprises identifying, for each phage in the phage prescription, at least one (and typically a plurality of) candidate phage vials in the phage storage apparatus that contain the respective phage.

We then determine a dispensing volume for one or more candidate phage vials based on a titer of the respective candidate phage vial such that the total dispensing volume from combining the one or more candidate phage vials will contain an amount of phage within the target phage dose range for the respective phage and an amount of contaminant, for each contaminant in the one or more contaminants, that is below the maximum contaminant level for the respective contaminant. The dispensing volume is based on the titer of the vial, which is expressed in PFU/mL. The target phage dose (or dose range) is given in units of PFU and thus a dispensing volume (e.g. the amount to draw into a syringe from the vial) is calculated from the titer by dividing the target dose by the titer (noting the titer is expressed in units of PFU/mL).

This may involve determining whether a single candidate phage vial has a titer such that a dispensing volume can be dispensed which will contains an amount of phage within the target dose range. In some embodiments if the titer is much larger than is required (e.g. such that the dispensing volume would be very small), then this may require diluting the phage. However if a single phage vial contains insufficient phage (titer too low) then a combination of candidate phage vials can be selected and combined such that the amount of phage in the total dispensing volume is within the target dose range. This determination is performed subject to the constraint that the amount of each of the one or more contaminants in the total dispensed volume is below the maximum contaminant level for the respective contaminant. In some embodiments this may be performed by performing a search to identify the set of one or more candidate phage vials which contain an amount of phage closest to the target phage dose.

After generation of the dispensing set and the associated dose calculation to determine the dispensing volumes, the user interface provides the storage location (e.g., box C2, bay 10) and dose preparation instructions 118 for each of the candidate phage vials in the dispensing set to the user. The dose preparation instructions comprise the dispensing volume (based on the titer) for each phage vial in the dispensing set along with any relevant instructions or direction on how to generate a dispensing dose for delivery to the patient. For example it may specify the dose is to be delivered by intravenous IV and provide instructions such as obtaining a 25 mL 0.9% saline minibag and then using a syringe, extract the dispensing dose from each vial and inject into a port in the saline bag or IV line to deliver to the dose to the patient. The prescription may also specify if any other pharmaceutical preparations are to be added.

The pharmacist may then remove a vial from the phage storage apparatus (e.g. a freezer, a refrigerator, or a cooler) 27. To ensure accurate dispensing the system then requires the pharmacist to input the phage vial identifier (ID), such as by reading (e.g. scanning) a barcode (1D or 2D) or reading a RFID tag located on the vial, or entering a serial number located on the phage vial 120 in order to ensure the correct vial is removed. The phage vial ID may be a serial number printed in a machine readable format, such as a barcode, on the vial or directly onto the vial which encodes the serial number. The phage vial ID may be stored in a machine readable format by an RFID tag which is attached or adhered to the vial, such as in the form of a label with an adhesive backing. The serial number may also be printed on the label or directly on the vial along with the barcode representing the phage vial identifier (e.g., serial number) in machine readable form, or just the barcode may be printed onto the label or vial. The barcode may be a 1D or linear barcode, of a 2D barcode such as a Quick Response (QR) code, PDF417 a Data Matrix, Aztec, Codablock-F, MaxiCode, MicroPDF417, Han Xin, Dot Codes, and variants such as SnapCodes, and High Capacity Color Barcodes. The barcode may encode a phage vial identifier such as a serial number, along with additional information such as a Uniform Resource Locator (URL) or Uniform Resource Identifier (URI) link to a webpage or document with details of the vial contents such as batch, titer, contaminants, or a certificate of analysis. In one embodiment the RFID tag is a passive tag comprising a microchip or integrated circuit, an antenna and a substrate with an adhesive backing allowing it to be used as a label which is stuck on the vial. The RFID tag may operate in the UHF range (e.g. 865 MHz to 928 MHZ) and operate according to the ISO/IEC 18000 standard. The microchip or integrated circuit may store the phage vial ID and transmit the phage vial ID as part of a reply message when the RFID tag is read (i.e. receives an interrogator signal). In some embodiments the phage vial identifier is a factory assigned tag ID (e.g. assigned by the RFID tag manufacturer), and when the label is attached to the vial the tag ID is associated (e.g. is used as a lookup key) with a phage record of a database. In this embodiment the reader apparatus reads the tag ID and then uses the tag ID to lookup the phage record. In one embodiment, the phage vial ID is a separate serial number to the tag ID which is stored in the phage record, and the factory assigned tag ID is used to obtain the phage vial ID (e.g. by looking up the phage vial ID in the phage record. In some embodiments the RFID tag stores additional information regarding the vial such as lot number, date of manufacture, etc. which is included in the reply message. In some embodiments active RFID tags may be used. In some embodiments the RFID tag and adhesive are formed of materials suitable for low temperature storage. The phage dispensing application 22 thus receives a phage vial identifier (ID) obtained (e.g. read, scanned, manually input, or otherwise input or obtained) from the removed phage vial, for example via reader apparatus 26. In one embodiment the reader apparatus is a barcode scanner comprising a light source and light sensor, or a camera of the computing apparatus 21 and associated software configured to extract (read, scan or recognize) the barcode from a captured image. In another embodiment the reader apparatus 26 is an RFID reader, which transmits interrogatory signals and receives a reply signal (or message) from an RFID tag on the vial (or adhered to the vial) which includes the phage vial ID or includes data from which the phage vial ID may be obtained, such as by looking up in a database record. In the case the barcode is damaged, or the reader apparatus 26 fails to read the barcode, the user interface may be configured to receive the phage vial identifier by another user interface device, such as by the user manually typing the serial number using a keyboard or touchscreen, or speaking the serial number into a microphone which captures the audio and converts the audio to a serial number. The read/scanned phage vial ID is then checked (compared) to ensure that it matches a stored phage vial ID of one of the phage vials in the dispensing set. If the phage vial ID matches (i.e., the correct vial has been removed) then the electronic phage inventory is updated 122 to record the dispensing of the phage vial. Confirmation of removal of a phage vial may further trigger a request for new stock (resupply) 124 by the electronic phage inventory. However, if there is no match then a warning is used to the user, for example to double check the correct vial has been removed. Once all phage vials have been read/scanned and approved, the pharmacist can use the phage vials and instructions to prepare the phage treatment, which is then dispensed to the clinician 126 for treating the patient. The electronic phage inventory may be locally maintained by the phage dispensing application 22, or the phage dispensing application 22 may send the confirmation of phage dispensing to the phage management application 62 executing on a remote server (including a cloud server) or to a database associated with the phage management application 62, which maintains the local inventory.

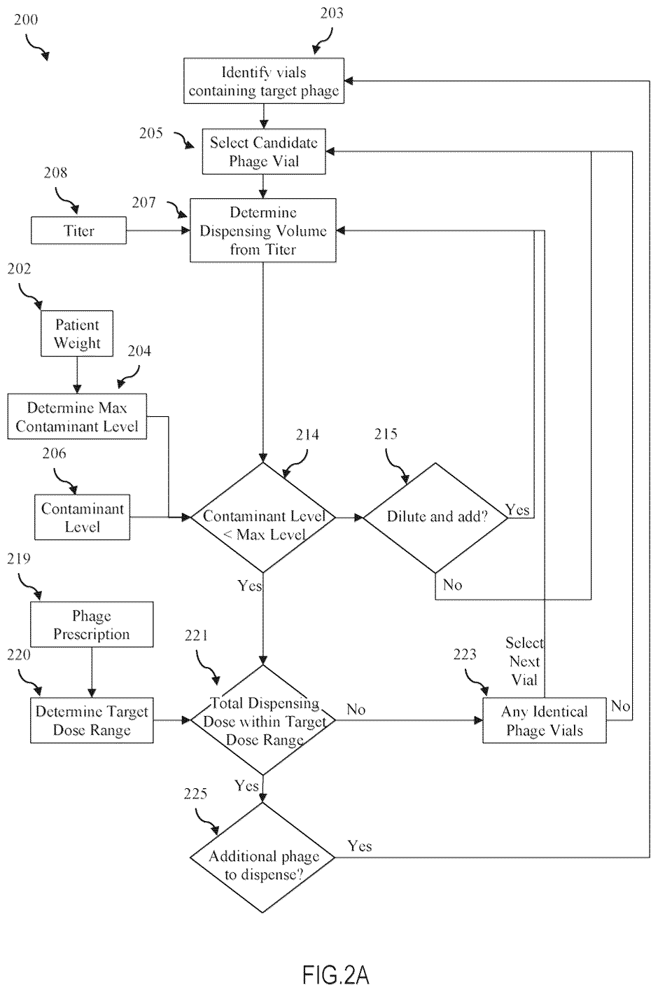

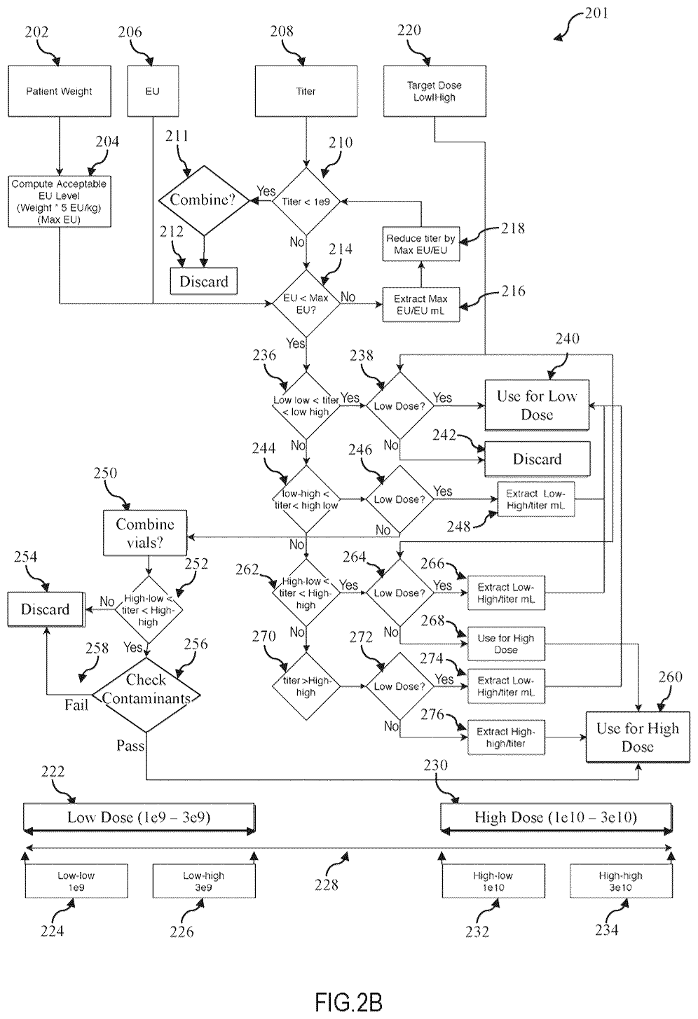

FIG. 2A is a flowchart of a dispensing method 200 according to an embodiment. As noted above the patient weight 202 is used to determine the maximum contaminant level 204 and the phage prescription 219 is used to determine a target dose range 220 for a phage. Generation of the dispensing set comprises identifying vials containing the target phage 203, selecting a first candidate phage vial 205 and determining a dispensing volume 207 based on the titer 208 of the first candidate phage vial. A test 214 may also be performed to check that the contaminant level 206 of the vial is less than the maximum contaminant level 204. If not then we calculate if the vial can be diluted to reduce the contaminant level below the maximum 215, and if so add the vial to the dispensing set and recalculate the dispensing volume 207, otherwise we can discard and select another candidate phage vial 205. If the dispensing volume is less than the volume required to dispense the phage prescription for the respective phage 221, then we perform a search for one or more additional candidate phage vials which are identical phage vials to the first candidate phage vial 223. An identical phage vial is a vial with the same titer and contaminant levels, or their values are within a predefined range of each other (e.g. 0.1%, 0.5%, 1%, 2%, 5% or 10%). Alternatively (or additionally), an identical phage vial may be a phage vial grown in the same batch, or grown in the same batch and undergone the same post batch processing (e.g. purification and dispensing into a vial) such that the vial was filled and sealed on the same day or within a predefined time limit (e.g. within 12, 24 or 48 hours). Such vials may be assigned the same lot number and thus vials with the same lot number are considered identical phage vials and determining if two phage vials are identical comprises checking if they have the same lot number.

As the phage are identical the known titer 202 and contaminant levels 206 can be used to determine how many additional candidate phage vials are required to generate the required total dispensing volume whilst ensuring that the contaminant levels remain below the maximum value. If the search identified sufficient identical candidate phage vials, then these are progressively added to the dispensing set, along with the associated dispensing volume 223.For example if the vials are 1 ml vials, and the total dispensing dose was 3.6 ml, then taking into account 1 mL from the first candidate vial, we would select 3 additional identical candidate vials, with associated dispensing volumes of 1 mL, 1 mL and 0.6 mL from the second, third and fourth candidate vials.

If an insufficient number of identical candidate phage vials are located to enable dispensing of the total dispensing dose, then we may select all of the identical candidate phage vials located and add them to the dispensing set along with an associated dispensing volume set to their maximum dispensing volume. For example in the previous case, if only two identical vials were located, we would add them both with dispensing volumes of 1 mL and 1 mL. If there are none found, we move to the next candidate phage vial 205.

We then repeat the above process (e.g. Loop 205 to 221) as many times as needed for each additional phage 225 until the dispensing set contains sufficient candidate phage vials to enable dispensing of the total dispensing volume 221 whilst ensuring that the contaminant levels remain below the maximum contaminant level (for each contaminant) 214. That is we identify another candidate phage vial 205 containing the target phage and determine the dispensing volume from the titer 207 and check the contaminant level(s) 214 are still below the maximum, and if so add this to the dispensing set. If there is still insufficient vials to enable dispensing of the total dispensing volume then we perform another search for identical candidate phage vials (to this additional candidate phage vial), and determine if these additional identical candidate phage vials can be added. Candidate vials with contaminant levels, which when added to the dispensing set, would result in the maximum contaminant level being exceeded may be discarded from the search, and alternative candidate phage vials identified.

In one embodiment we may first identify sets of identical phage vials, and identifying at least one candidate phage vials for a phage may involve sequentially selecting a candidate phage vial from each identical set, and then using the rest of the phage vials in the identical set as required.

In one embodiment the search may involve searching for a combination of candidate phage vials which when combined generate a total dose closest to a target dose within the target dose range. This may be subject to a constraint on the combination with the minimum contaminant levels. In one embodiment an optimization method may be used to search for an optimal combination that is closest to the target phage dose whilst minimizing contaminants. For example if the optimizer minimizes a cost function (such that low costs are better) the cost function could include a dose term which uses a parabolic or V shaped weight function centered on the target dose range and which becomes infinite or very large outside of the target phage dose range limits and a term for each contaminant which increases linearly or exponentially up to the maximum contaminant level after which it becomes infinite or very large. In the case of multiple contaminants, different contaminants could use different weights to priorities the importance of different contaminants.

The above methodology can be extended to phage prescription comprising multiple phage. The search for candidate phage vials may be performed sequentially for each phage in the prescription. Alternatively the search may be performed in parallel to identify combination of vials which contain dispensing volumes for each of the respective phage, and for which the total contaminant levels are below the maximums. Optimization methods discussed above can be used for parallel searching.

FIG. 2B is a flowchart of a dose calculation method 201 for a candidate phage vial used to generate the dispensing set according to another embodiment. The dose calculation method 201 calculates a dispensing volume, based on the titer, for a candidate phage vial, or combination of candidate phage vials, or determines that a candidate phage vial is unsuitable for dispensing to the patient due to excessive contaminants (alone or when combined with other candidate phage vials). The dose calculation begins with the patient weight 202, the target phage dose range 220, a titer of the candidate phage vial 208 and one or more contaminant levels which in this embodiment is a single contaminant in the form of an endotoxin (EU) level of the candidate phage vial 206. In this embodiment the target phage dose range 220 is either a low dose range 222 or a high dose range 230. The low dose range 222 has a range from a LowDose-low limit 224 to a LowDose-high limit 226 as shown at the bottom of FIG. 2B. In this embodiment the Low Dose-low limit is 1e9 PFU/ml and the Low Dose-high limit is 3c9 PFU/ml (a PFU is a plaque forming unit). The high dose 230 has a high dose range from a HighDose-low limit 232 to a HighDose-high limit 234. In this embodiment the HighDose-low limit 232 is 1e10 PFU/ml and the HighDose-high limit 234 is 3e19 PFU/ml. The full dosage range 228 is thus from the LowDose-low limit 224 to HighDose-high limit 234.

An acceptable endotoxin (EU) level is calculated at 204 by multiplying the patient weight by 5 EU/kg to obtain a Max EU level-the maximum acceptable endotoxin level. We then compare 214 the endotoxin level 206 of the candidate phage vial (obtained from the certificate of analysis) to the maximum acceptable endotoxin level (max EU) 214. If the endotoxin level of the candidate phage vial exceeds the calculated maximum acceptable endotoxin level (EU>Max EU) then at 216 we determine a reduced titer level 218 by multiplying the titer 208 of the candidate phage vial by the ratio of the maximum acceptable endotoxin level 204 to the endotoxin level 206 of the candidate phage vial. We then check if the reduced titer level exceeds the minimum dose titer 210. If the reduced titer level is less than the minimum dose (the LowDose-low dose 1e9) 210 then we can either determine if the diluted phage vial can be combined with another candidate phage vial to deliver a total dose in the target range, or we can reject and discard the candidate phage vial 212. If the reduced titer level exceeds the minimum dose we use the reduced titer level calculated at 218 in place of the actual titer level 208 of the candidate phage vial for the rest of the dose calculation. Similarly, if the titer 208 of the candidate phage vial is less than the minimum dose then we can either determine if the diluted phage vial can be combined with another candidate phage vial to deliver a total dose in the target range, or we can reject and discard the candidate phage vial 212.

If the titer exceeds the minimum dose and the endotoxin level is less than the maximum acceptable endotoxin level we then check 236 if the titer of the candidate phage vial is between the Low Dose-low limit and the Low Dose-high limit. If the target dose is the low dose 238 then we use the titer 208 of the candidate phage vial as the dose titer 240 and add the candidate phage vial to the dispensing set. If the target dose is the high dose then we reject the candidate phage vial 242 as not suitable for the patient.

We then check 244 if the titer of the candidate phage vial is between the Low Dose-high limit and the HighDose-low limit. If yes and the target dose is the low dose 246 then we set the dose titer to the LowDose-high titer and add the candidate phage vial to the dispensing set 248. If the target dose is the high dose 264 we then check 250 if the candidate phage vial can be combined with a second candidate phage vial to obtain a titer in the high dose range 252 (i.e., between HighDose-low and HighDose-high). If the dose is outside this range, we reject the candidate phage vial 254 as not suitable for the patient. We also check that one or more excipients are in an acceptable range 256. If not 258 (fail) we reject the candidate phage vial 254 as not suitable for the patient and if yes (pass) we add the candidate phage vial and the second candidate phage vial to the dispensing set for use as the high dose 260.

If check 244 returned no (the titer of the candidate phage vial is now between the LowDose-high limit and the HighDose-low limit), we next check 262 if the titer of the candidate phage vial is between the HighDose-low limit and the HighDose-high limit and if yes and the target dose is the low dose 264 then we set the dose titer to the Low Dose-high titer 266 and add the candidate phage vial to the dispensing set. If the target dose is the high dose 268 then we use the titer of the candidate phage vial as the dose titer and add the candidate phage vial to the dispensing set.

If check 262 returned no then the titer of the candidate phage vial exceeds the HighDose-high limit (this can be explicitly checked) 270 and if the target dose is the low dose 272 then we set the dose titer to the Low Dose-high titer 274 and add the candidate phage vial to the dispensing set and if the target dose is the high dose 276 then we set the dose titer to the HighDose-high titer and add the candidate phage vial to the dispensing set.

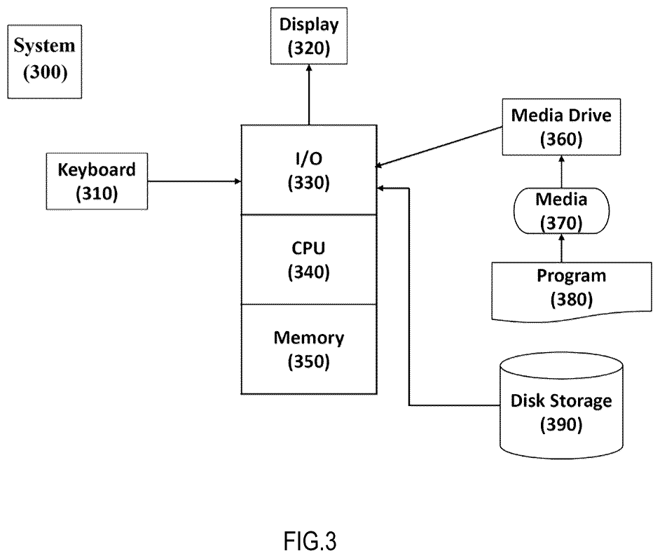

FIG. 3 depicts an exemplary computing system configured to perform any one of the computer implemented methods described herein. The computing system may comprise one or more processors including multi-core CPUs and Graphical Processing Units (GPUs) operatively connected to one or more memories which store instructions to configure the processor to perform embodiments of the method. In this context, the computing system may include, for example, one or more processors (CPUs, GPUs), memories, storage, networking interfaces and input/output devices (e.g., monitor, keyboard, disk drive, network interface, Internet connection, etc.). However, the computing system may include circuitry or other specialized hardware for carrying out some or all aspects of the processes. The computing system may be a computing apparatus such as an all-in-one computer, desktop computer, laptop, tablet or mobile computing apparatus, server, and any associated peripheral devices. The computer system may be a distributed system including server-based systems and cloud-based computing systems. In some operational settings, the computing system may be configured as a system that includes one or more units, each of which is configured to carry out some aspects of the processes either in software, hardware, or some combination thereof. For example, the user interface may be provided on a desktop computer or tablet computer, whilst the dose calculations may be performed on a server-based system including cloud-based server systems, and the user interface is configured to communicate with such servers. The user interface may be provided as a web portal, allowing a user on one computer to upload data (e.g. user ID, site ID, patient ID) which may be processed on a remote computing apparatus or system (e.g., server or cloud system) and which provides the results (i.e., the dispensing instructions) back to the user, or to other users on other computing apparatus.

FIG. 3 depicts an exemplary computing system (300) with a number of components that may be used to perform the processes described herein. For example, an input/output (“I/O”) interface 330, one or more central processing units (“CPU”) (340), and a memory section (350). The I/O interface (330) is connected to input and output devices such as a display (320), a keyboard (310), a disk storage unit (390) such as a SSD, and a media drive unit (360) configured to read/write a removable computer-readable medium (370), which can contain programs (380) and/or data. The disk storage unit (390) may also be used to store programs (380) and/or data, including programs or data downloaded from a website or read from media inserted in the media drive unit. The I/O interface may comprise a network interface and/or communications module for communicating with an equivalent communications module in another device using a predefined communications protocol (e.g., Bluetooth, Zigbee, IEEE 802.15, IEEE 802.11, TCP/IP, UDP, etc.). A computer program or application may be written, for example, in a general-purpose programming language (e.g., Swift, Python, Java, C, C++, C#, Pascal, JSON, etc.) or some specialized application-specific language to implement the system or components and may call software libraries/packages.

The dose preparation instructions are displayed on the screen and may also be printed by printer 25 and/or emailed to the pharmacist. For example, the dosing instructions may include patient ID, phage vial location (C2, bay 10), target dose (e.g., low dose), the dispensing volume and preparation instructions such as
“Remove vial from the freezer location”;
“Touch camera icon to scan the vial's barcode”;
“withdraw 1.00 mL from the vial”; and
“then inject volume into a 25 mL minibag (NS).

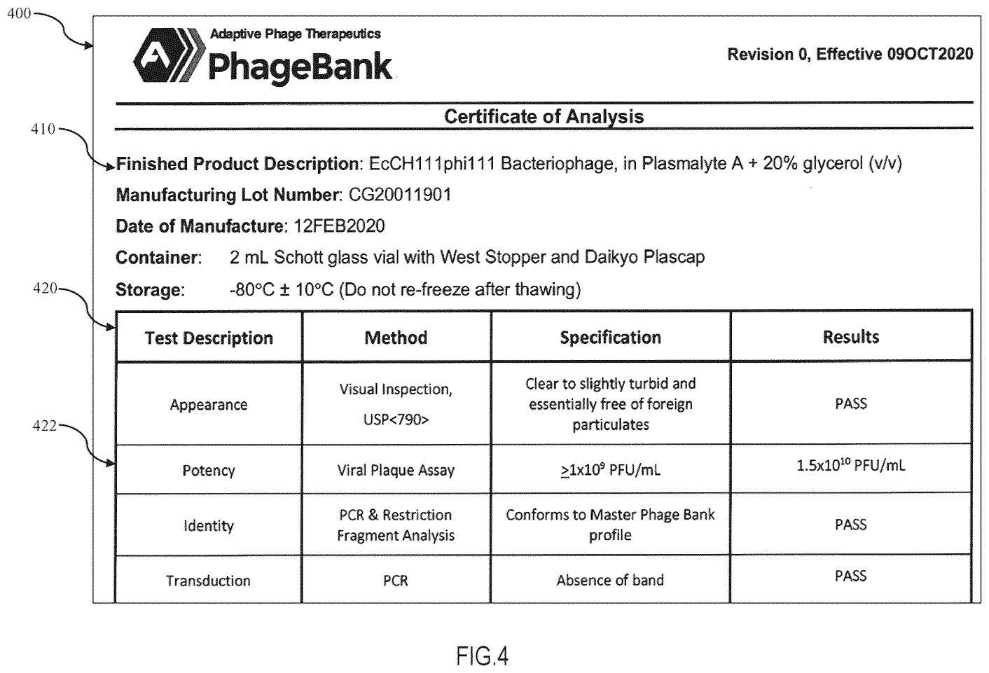

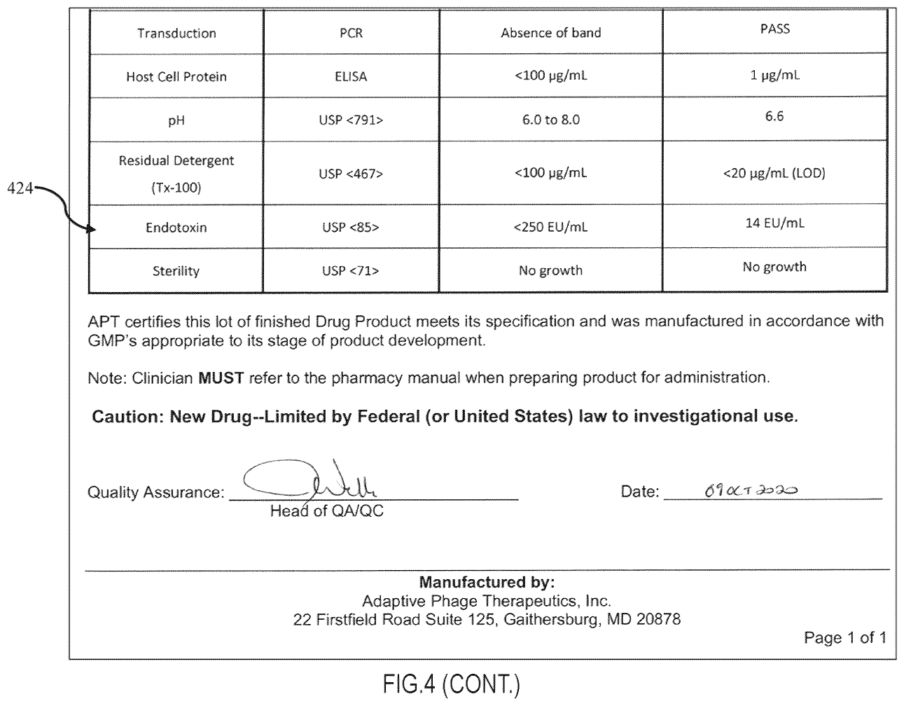

The electronic certificate of analysis for each of the phage vials in the dispensing set may also be displayed to the pharmacist. FIG. 4 is a representation of a certificate of analysis 400 of a phage vial according to an embodiment. This may be generated by quality control laboratory 54 of the manufacturer 50 and an electronic copy stored by the phage management system 60. The certificate of analysis comprises manufacturing data 410 such as the product description, manufacturing lot number and date of manufacturer. Details of the storage vial (Schott glass vial) and storage conditions (−80° C.) may also be provided. A test description table 420 includes details on tests such as appearance, potency (to obtain the titer 422), identity, transduction, pH, sterility and contaminant tests/levels such as host cell protein levels, residual detergent, and endotoxin level 424. The data from the certificate of analysis may be stored in a phage record by phage management system 60.

The phage management system 60 may also perform administrative tasks and may include a traceability database that logs activities such as user logins (time and IP address) and actions, such as patients selected, which boxes were accessed by a pharmacist, and barcode scans, RFID reads, weight data and weight changes, app alerts, and whether the user has pressed buttons to print or send dose instructions or view the certificate of analysis. The phage management system may also coordinate exchange of data between the various applications at different sites. In one embodiment the phage management system is further configured to interface or communicate with LIMS and/or EHR systems to look up data or record the dispensing of the prescription to the patient.

Embodiments of the system and method provide a more accurate and efficient dispensing system for phage based treatments, thus assisting pharmacists to accurately dispense phage based treatments. The system also monitors dispensing and coordinates data exchanges between various distributed components to ensure supplies of phage are maintained at dispensing sites.

The system and method allows personalized and dynamic dosing instructions to be delivered to the pharmacist based on the specific phage treatment in order to effectively treat the patient. Pharmacists are not accustomed to the drug product being different from type to type (e.g., due to batch to batch variability) and thus the system and method is configured to both guide the pharmacist and checks their activities to ensure accurate and efficient dispensing. The system also provides a detailed audit trail to assist in meeting any traceability and auditability requirements (e.g., FDA 21 part 11) which is more reliable than current paper and email-based methods.

The steps of a method or algorithm described in connection with the embodiments disclosed herein may be embodied directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors including CPU and GPUS, controllers, micro-controllers, microprocessors, other electronic units designed to perform the functions described herein, or a combination thereof. Software modules, also known as computer programs, computer codes, or instructions, may contain a number of source code or object code segments or instructions, and may reside in any computer readable medium such as a memory (including RAM, flash, ROM, EPROM, registers, etc.), hard disks (including SSDs), networked storage, a removable disk, a CD-ROM, a DVD-ROM, a Blu-ray disc, or any other form of computer readable medium. In some aspects the computer-readable media may comprise non-transitory computer-readable media (e.g., tangible media). In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

A non-transitory computer-program product or storage medium comprising computer-executable instructions for carrying out any of the methods described herein can also be generated. A non-transitory computer-readable medium can be used to store (e.g., tangibly embody) one or more computer programs for performing any one of the above-described processes by means of a computer. Further provided is a computer system comprising one or more processors, memory, and one or more programs, wherein the one or more programs are stored in the memory and configured to be executed by the one or more processors, the one or more programs including instructions for carrying out any of the methods described herein.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A computer implemented method for dispensing a phage treatment, the method comprising:
receiving electronically phage information for a patient at a site for treatment, the phage information comprising one or more phage to be dispensed at the site;
generating a dispensing set comprised of one or more phage vials stored in a phage storage apparatus by
determining, for each phage in the phage information, one or more candidate phage vials in the phage storage apparatus that contain the respective phage; and
providing a storage location for each of the one or more candidate phage vials in the dispensing set to generate the phage treatment for the patient at the site.

2. The computer implemented method as claimed in claim 1, wherein the phage information comprises a target phage dose for each of the one or more phages to be dispensed and a target phage dose range for each of the one or more phages to be dispensed is determined from the respective target phage dose.

3. (canceled)

4. (canceled)

5. (canceled)

6. The computer implemented method as claimed in claim 1, further comprising determining a dispensing volume for one or more candidate phage vials by determining a dispensing volume for a first candidate phage vial using a titer of the first candidate phage vial, and when the dispensing volume is less than a volume required to dispense the phage treatment for the respective phage, then searching for one or more additional candidate phage vials which are identical phage vials to the first candidate phage vial, and when one or more identical phage vials to the first phage candidate phage vial are located, then adding one or more of the one or more identical phage vials to the one or more candidate phage vials and storing an associated dispensing volume for each of the one or more identical phage vials when an amount of contaminant, for each of one or more contaminants, is below a maximum contaminant level for the respective contaminant, and when the dispensing set does not comprise sufficient candidate phage vials and associated dispensing volumes to enable dispensing of the phage treatment for the respective phage, then repeating until the dispensing set comprises sufficient candidate phage vials to dispense the phage treatment for the respective phage such that the amount of each contaminant is below the maximum contaminant level for the respective contaminant.

7. (canceled)

8. (canceled)

9. The computer implemented method as claimed in claim 1, wherein the method further comprises determining a target phage dose for each of the one or more phages to be dispensed and using an optimization method to search for a combination of candidate phage vials and associated dispensing volumes which when combined generate a total dose closest to the target phage dose subject to a constraint that an amount of contaminant, for each of one or more contaminants, is below a maximum contaminant level for the respective contaminant.

10. (canceled)

11. The computer implemented method as claimed in claim 1, wherein a computing apparatus is operatively connected to the phage storage apparatus, a phage vial identifier is a barcode or a radio frequency identification tag, and the method further comprises;
requesting, via the computing apparatus, a user enter a patient weight;
on receiving the patient weight, generating, via the computing apparatus, the dispensing set and displaying the storage location and dose preparation instructions for each of the candidate phage vials in the dispensing set on a display device; and reading the barcode or the radio frequency identification tag of a vial to verify the vial belongs to the dispensing set.

12. The computer implemented method as claimed in claim 1, wherein providing a storage location further comprises displaying, printing, and/or emailing the storage location and dose preparation instructions to a user.

13. (canceled)

14. (canceled)

15. (Canceled)

16. The computer implemented method as claimed in claim 1, wherein when a phage vial identifier obtained from a phage vial matches a stored phage vial identifier of one of the phage vials in the dispensing set, updating an electronic phage inventory to record dispensing of the phage vial, and sending a request to a remote store to ship additional phage vials to the phage storage apparatus.

17. A phage dispensing apparatus comprising:
at least one memory, and
at least one processor, wherein the at least one memory comprises instructions to configure the at least one processor to:
receive electronically phage information for a patient at a site for treatment, the phage information comprising one or more phage to be dispensed at the site;
generate a dispensing set comprised of one or more phage vials stored in a phage storage apparatus by
determining, for each phage in the phage information, one or more candidate phage vials in the phage storage apparatus that contain the respective phage; and
provide a storage location for each of the one or more candidate phage vials in the dispensing set to generate a phage treatment for the patient at the site.

18. The phage dispensing apparatus as claimed in claim 17, wherein the phage information comprises a target phage dose for each of the one or more phages to be dispensed and a target phage dose range for each of the one or more phages to be dispensed is determined from the respective target phage dose.

19. (canceled)

20. (canceled)

21. (canceled)

22. The phage dispensing apparatus as claimed in claim 17, wherein the at least one processor is configured for determining a dispensing volume for one or more candidate phage vials by determining a dispensing volume for a first candidate phage vial using a titer of the first candidate phage vial, and when the dispensing volume is less than a volume required to dispense the phage treatment for the respective phage, then searching for one or more additional candidate phage vials which are identical phage vials to the first candidate phage vial and when one or more identical phage vials to the first phage candidate phage vial are located, then adding one or more of the one or more identical phage vials to the one or more candidate phage vials and storing an associated dispensing volume for each of the one or more identical phage vials when an amount of contaminant, for each of one or more contaminants, is below a maximum contaminant level for the respective contaminant, and when the dispensing set does not comprise sufficient candidate phage vials and associated dispensing volumes to enable dispensing of the phage treatment for the respective phage, then repeating until the dispensing set comprises sufficient candidate phage vials to dispense the phage treatment for the respective phage such that the amount of each contaminant is below the maximum contaminant level for the respective contaminant.

23. (canceled)

24. (canceled)

25. The phage dispensing apparatus as claimed in claim 17, wherein the at least one processor is further configured to determine a target phage dose for each of the one or more phages to be dispensed and use an optimization method to search for a combination of candidate phage vials and associated dispensing volumes which when combined generate a total dose closest to the target phage dose subject to a constraint that an amount of contaminant, for each of one or more contaminants, is below a maximum contaminant level for the respective contaminant.

26. (canceled)

27. The phage dispensing apparatus as claimed in claim 17, wherein the phage dispensing apparatus is operatively connected to the phage storage apparatus, a phage vial identifier is a barcode or a radio frequency identification tag and the phage dispensing apparatus further comprises;
at least one display device;
one or more user interface devices; and
a reader apparatus configured to read the barcode and/or a the radio frequency identification tag;
wherein the one or more user interface devices are configured to request a user enter a patient weight, and on receiving the patient weight the phage dispensing apparatus generates the dispensing set and displays the storage location and dose preparation instructions for each of the candidate phage vials in the dispensing set on the at least one display device, and the reader apparatus obtains the phage vial identifier by reading the barcode or the radio frequency identification tag of a vial to verify the vial belongs to the dispensing set.

28. The phage dispensing apparatus as claimed in claim 17, wherein providing a storage location further comprises displaying, printing, and/or emailing the storage location and dose preparation instructions to a user.

29. (canceled)

30. (canceled)

31. (canceled)

32. The phage dispensing apparatus as claimed claim 17, wherein the instructions further configure the at least one processor to:
when a phage vial identifier obtained from a phage vial matches a stored phage vial identifier of one of the phage vials in the dispensing set, update an electronic phage inventory to record dispensing of the phage vial, and send a request to a remote store to ship additional phage vials to the phage storage apparatus.

33. A non-transitory, computer program product comprising computer executable instructions for dispensing a phage treatment, the instructions executable by a computer to:
receive electronically phage information for a patient at a site for treatment, the phage information comprising one or more phage to be dispensed at the site;
generate a dispensing set comprised of one or more phage vials stored in a phage storage apparatus by
determining, for each phage in the phage information, one or more candidate phage vials in the phage storage apparatus that contain the respective phage included in the phage information; and
provide a storage location for each of the one or more candidate phage vials in the dispensing set to generate the phage treatment for the patient at the site.

34. (canceled)

35. (canceled)

36. (canceled)

37. (canceled)

38. The non-transitory, computer program product as claimed in claim 33, wherein the phage information comprises a target phage dose for each of the one or more phages to be dispensed and a target phage dose range for each of the one or more phages to be dispensed is determined from the respective target phage dose.

39. The non-transitory, computer program product as claimed in claim 33, wherein the instructions are executable by the computer for determining a dispensing volume for one or more candidate phage vials by determining a dispensing volume for a first candidate phage vial using a titer of the first candidate phage vial, and when the dispensing volume is less than a volume required to dispense the phage treatment for the respective phage, then searching for one or more additional candidate phage vials which are identical phage vials to the first candidate phage vial and when one or more identical phage vials to the first phage candidate phage vial are located, then adding one or more of the one or more identical phage vials to the one or more candidate phage vials and storing an associated dispensing volume for each of the one or more identical phage vials when an amount of contaminant, for each of one or more contaminants, is below a maximum contaminant level for the respective contaminant, and when the dispensing set does not comprise sufficient candidate phage vials and associated dispensing volumes to enable dispensing of the phage treatment for the respective phage, then repeating until the dispensing set comprises sufficient candidate phage vials to dispense the phage treatment for the respective phage such that the amount of each contaminant is below the maximum contaminant level for the respective contaminant.

40. The non-transitory, computer program product as claimed in claim 33, wherein the instructions are further executable by a computer to determine a target phage dose for each of the one or more phages to be dispensed and use an optimization method to search for a combination of candidate phage vials and associated dispensing volumes which when combined generate a total dose closest to the target phage dose subject to a constraint that an amount of contaminant, for each of one or more contaminants, is below a maximum contaminant level for the respective contaminant.

41. The non-transitory, computer program product as claimed in claim 33, wherein providing a storage location further comprises displaying, printing, and/or emailing the storage location and dose preparation instructions to a user.

42. The non-transitory, computer program product as claimed claim 33, wherein the instructions are further executable by the computer to:
when a phage vial identifier obtained from a phage vial matches a stored phage vial identifier of one of the phage vials in the dispensing set, update an electronic phage inventory to record dispensing of the phage vial, and send a request to a remote store to ship additional phage vials to the phage storage apparatus.
